(19) United States
(12) Patent Application Publication
(10) Pub. No.: US 2009/0247750 A1
(43) Pub. Date: Oct. 1, 2009

(54) PROCESS FOR PREPARING NUCLEOSIDE ANALOGS

(75) Inventors: Pravin L. Kotian, Birmingham, AL (US); Kailas B. Sawant, Birmingham, AL (US); Pooran Chand, Birmingham, AL (US)

Correspondence Address: VIKSNINS HARRIS & PADYS PLLP, P.O. BOX 111098, ST. PAUL, MN, 55111-1098, US

(73) Assignee: BioCryst Pharmaceuticals, Inc., Birmingham, AL (US)

(21) Appl. No.: 12/413,119

(22) Filed: Mar. 27, 2009

Related U.S. Application Data
(60) Provisional application No. 61/040,226, filed on Mar. 28, 2008.

(51) Int. Cl. C07D 487/04

(52) U.S. Cl. 544/280

(57) ABSTRACT

The invention provides an improved method for preparing a compound of formula 2:

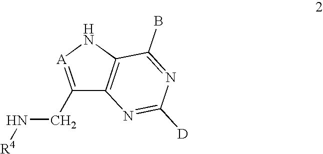

that is useful to treat diseases wherein it is desirable to inhibit purine nucleoside phosphorylases or nucleoside hydrolases (e.g., parasitic infections, inflammatory disorders, etc.).

### RELATED APPLICATION(S)

This patent application claims the benefit of priority of U.S. application Ser. No. 61/040,226, filed Mar. 28, 2008, which application is herein incorporated by reference.

### BACKGROUND

International Patent Application Publication Number WO 2008/030119 discloses the following compounds of formula (I), which are reported to be useful to treat diseases wherein it is desirable to inhibit purine nucleoside phosphorylases or nucleoside hydrolases (e.g., parasitic infections, inflammatory disorders, etc.):

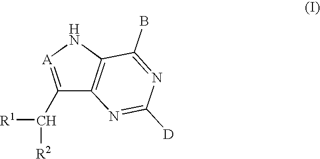

wherein:

R¹ is H or NR³R⁴;

R² is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, amino, hydroxy, or alkoxy groups; provided that when R¹ is H, R² is an alkyl, alkenyl, alkynyl, aralkyl, aralkenyl, aralkynyl, or aryl group which is substituted with at least one NR³R⁴ group;

provided that when R¹ is H, R² is alkyl, alkenyl, alkynyl, aralkyl, aralkenyl, aralkynyl, or aryl group which is substituted with at least one NR³R⁴ group;

R³ and R⁴, independently of each other, is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, amino, hydroxy, or alkoxy groups;

A is N or CH;

B is OH or alkoxy; and

D is H, OH, NH₂, or SCH₃;

provided that when R¹ is NR³R⁴, R² is H, A is CH, B is OH, and D is H, then R³ is not hydroxyethyl or hydroxypropyl when R⁴ is hydroxyethyl; and

provided that when R¹ is NR³R⁴, R² is H, A is CH, B is OH, and D is NH₂, then R³ is not hydroxyethyl when R⁴ is H, methyl, ethyl, or hydroxyethyl; and R⁴ is not hydroxyethyl when R³ is H, methyl, ethyl, or hydroxyethyl;

or a tautomer thereof, or a pharmaceutically acceptable salt thereof, or an ester prodrug form thereof.

At page 17 of International Patent Application Publication Number WO 2008/030119, there is a discussion regarding the use of a Mannich reaction or reductive amination in the preparation of compounds of formula (I). Additionally, at Example 16 therein, a Mannich reaction is used to prepare a compound of formula 31.3 in a reported 50% yield.

The Mannich reaction is a classic method for the preparation of β-amino ketones and aldehydes. Unfortunately, this classic reaction is plagued by a number of serious disadvantages. Due to the drastic reaction conditions, single products are generally only obtained when secondary amines are used (M. Arend et al., Angewandte Chemie. Int. Ed. 1998, 37, 1044-1070). As a result, one would typically not select a Mannich reaction to prepare a compound of formula (I) wherein R¹ is NR³R⁴ and one of R³ and R⁴ is hydrogen on a commercial scale (e.g., multi-gram or kilo-gram scale). Because the preparation of the aldehyde starting material for the reductive amination reaction is time consuming and requires four steps, there is a need for improved methods for preparing the compounds of Formula (I) wherein R¹ is NR³R⁴ and one of R³ and R⁴ is hydrogen, particularly on a commercially useful scale.

### SUMMARY OF THE INVENTION

Unexpectedly, it has been determined that the compounds of formula (I) wherein R¹ is NR³R⁴ and one of R³ and R⁴ is hydrogen can be prepared in commercially viable yields (e.g., at least about 60%) employing a Mannich reaction in a suitable solvent system. Accordingly in one embodiment, the invention provides a method for preparing a compound of formula 2:

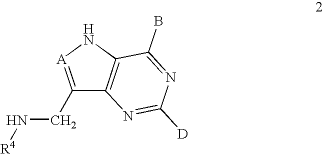

wherein:

R⁴ is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR^{a}R^{b} groups, where each alkylthio, arylthio and aralkylthio group is optionally substituted with one or more alkyl, halogen, amino, hydroxy, or alkoxy groups; or R⁴ is such a group bearing one or more protecting groups;

A is N or CH;

B is OH or alkoxy;

D is H, OH, NH₂, or SCH₃; and

R^{a} and R^{b} are each independently an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, or nitro;

comprising reacting a corresponding compound of formula 3

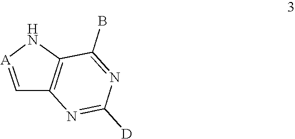

with a corresponding amine of formula R⁴NH₂ and a formaldehyde source in a suitable solvent to provide a reaction mixture comprising the compound of formula 2 in at least about 60% percent yield based on the starting compound of formula 3.

In another embodiment the invention provides a method for preparing a salt of a compound of formula 2:

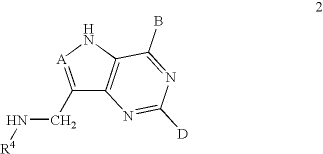

wherein:

R⁴ is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR^{a}R^{b} groups, where each alkylthio, arylthio and aralkylthio group is optionally substituted with one or more alkyl, halogen, amino, hydroxy, or alkoxy groups; or R⁴ is such a group bearing one or more protecting groups;

A is N or CH;

B is OH or alkoxy;

D is H, OH, NH₂, or SCH₃; and

R^{a} and R^{b} are each independently an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, or nitro;

comprising reacting a corresponding compound of formula 3

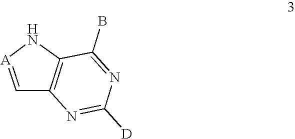

with a corresponding amine of formula R⁴NH₂ and a formaldehyde source in a suitable solvent to provide a compound of formula 2 in a reaction mixture; and

combining a suitable acid with the reaction mixture to provide the salt of the compound of formula 2 in a second reaction mixture.

### DETAILED DESCRIPTION

The term “alkyl” means any saturated hydrocarbon radical having up to 30 carbon atoms and includes any C1-C25, C1-C20, C1-C15, C1-C10, or C1-C6 alkyl group, and is intended to include both straight- and branched-chain alkyl groups. The same terminology applies to the non-aromatic moiety of an aralkyl radical. Examples of alkyl groups include: methyl group, ethyl group, n-propyl group, iso-propyl group, n-butyl group, iso-butyl group, sec-butyl group, t-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group and 1-methyl-2-ethylpropyl group.

The term “alkenyl” means any hydrocarbon radical having at least one double bond, and having up to 30 carbon atoms, and includes any C2-C25, C2-C20, C2-C15, C2-C10, or C2-C6 alkenyl group, and is intended to include both straight- and branched-chain alkenyl groups. The same terminology applies to the non-aromatic moiety of an aralkenyl radical. Examples of alkenyl groups include: ethenyl group, n-propenyl group, iso-propenyl group, nbutenyl group, iso-butenyl group, sec-butenyl group, t-butenyl group, n-pentenyl group, 1,1-dimethylpropenyl group, 1,2-dimethylpropenyl group, 2,2-dimethylpropenyl group, 1-ethylpropenyl group, 2-ethylpropenyl group, n-hexenyl group and 1-methyl-2-ethylpropenyl group.

The term “alkynyl” means any hydrocarbon radical having at least one triple bond, and having up to 30 carbon atoms, and includes any C2-C25, C2-C20, C2-C15, C2-C10, or C2-C6 alkynyl group, and is intended to include both straight- and branched-chain alkynyl groups. The same terminology applies to the non-aromatic moiety of an aralkynyl radical. Examples of alkynyl groups include: ethynyl group, n-propynyl group, iso-propynyl group, n-butynyl group, iso-butynyl group, sec-butynyl group, t-butynyl group, n-pentynyl group, 1,1-dimethylpropynyl group, 1,2-dimethylpropynyl group, 2,2-dimethylpropynyl group, 1-ethylpropynyl group, 2-ethylpropynyl group, n-hexynyl group and 1-methyl-2-ethylpropynyl group.

The term “aryl” means an aromatic radical having 4 to 18 carbon atoms and includes heteroaromatic radicals. Examples include monocyclic groups, as well as fused groups such as bicyclic groups and tricyclic groups. Some examples include phenyl group, indenyl group, 1-naphthyl group, 2-naphthyl group, azulenyl group, heptalenyl group, biphenyl group, indacenyl group, acenaphthyl group, fluorenyl group, phenalenyl group, phenanthrenyl group, anthracenyl group, cyclopentacyclooctenyl group, and benzocyclooctenyl group, pyridyl group, pyrrolyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, furyl group, pyranyl group, benzofuryl group, isobenzofuryl group, thienyl group, thiazolyl group, isothiazolyl group, benzothiazolyl group, oxazolyl group, and isoxazolyl group.

The term “aralkyl” means an alkyl radical having an aryl substituent.

The term “alkoxy” means an hydroxy group with the hydrogen replaced by an alkyl group.

The term “halogen” includes fluorine, chlorine, bromine and iodine.

The term “optionally substituted” means, in reference to the optionally substituted group, the group may have one or more substituents chosen from the group comprising hydroxy, alkyl, alkoxy, thiol, optionally substituted alkylthio, optionally substituted arylthio, optionally substituted aralkylthio, halogen, amino, carboxylic acid, and carboxylate alkyl ester.

It will be appreciated by those skilled in the art that compounds having a chiral center may exist in and be isolated in optically active and racemic forms. Some compounds may exhibit polymorphism. It is to be understood that the present methods are directed to the preparation of any racemic, optically-active, polymorphic, or stereoisomeric form, or mixtures thereof, of a compound of Formula (I).

Specific values listed below for radicals, substituents, and ranges, are for illustration only; they do not exclude other defined values or other values within defined ranges for the radicals and substituents

A specific value for A is CH.

A specific value for B is OH.

A specific value for D is H.

A specific value for R⁴ is alkyl, alkenyl, or alkynyl, which is optionally substituted with one or more hydroxy, alkoxy, thiol, alkylthio, arylthio, aralkylthio, halogen, carboxylic acid, carboxylate alkyl ester, nitro, or NR^{a}R^{b} groups.

A specific value for R⁴ is alkyl, alkenyl, or alkynyl, which is optionally substituted with one or more hydroxy or alkoxy groups.

A specific value for R⁴ is alkyl which is optionally substituted with one or more hydroxy groups.

A specific value for R⁴ is —CH[CH₂OH][CH(OH)CH₂OH].

Any suitable source of formaldehyde can be used in the methods of the invention. For example, the formaldehyde source can be formalin or paraformaldehyde.

The reaction of the compound of formula 3 with an amine of formula R⁴NH₂ and formaldehyde can be carried out in any suitable solvent. For example, the solvent can comprises an alcohol, such as a (C₁-C₆)alcohol, e.g, tert-butanol or isopropanol.

The reaction of the compound of formula 3 with an amine of formula R⁴NH₂ and formaldehyde can be carried out at any suitable temperature, for example, at a temperature of 70±20° C. In one embodiment, the reaction is carried out at the reflux temperature of the solvent.

In one embodiment of the invention the reaction of the compound of formula 3 with an amine of formula R⁴NH₂ and a source of formaldehyde provides the compound of formula 2 in at least about 60 percent yield based on the starting compound of formula 3. In another embodiment of the invention the reaction of the compound of formula 3 with an amine of formula R⁴NH₂ and a source of formaldehyde provides the compound of formula 2 in at least about 70 percent yield based on the starting compound of formula 3. In another embodiment of the invention the reaction of the compound of formula 3 with an amine of formula R⁴NH₂ and a source of formaldehyde provides the compound of formula 2 in at least about 80 percent yield based on the starting compound of formula 3. In yet another embodiment of the invention the reaction of the compound of formula 3 with an amine of formula R⁴NH₂ and a source of formaldehyde provides the compound of formula 2 in at least about 85 percent yield based on the starting compound of formula 3.

In one embodiment of the invention, the reaction of the compound of formula 3 with an amine of formula R⁴NH₂ and a source of formaldehyde is carried out on a commercially useful scale. For example, in one embodiment of the invention, the reaction is carried out starting with at least about 1 gram of the compound of formula 3. In another embodiment of the invention, the reaction is carried out starting with at least about 10 grams of the compound of formula 3. In another embodiment of the invention, the reaction is carried out starting with at least about 100 grams of the compound of formula 3. In another embodiment of the invention, the reaction is carried out starting with at least about 500 grams of the compound of formula 3.

In one embodiment of the invention a suitable acid is added to the reaction mixture to provide a salt of the compound of formula 2 in a second reaction mixture. Suitable acids include acetic acid, adipic acid, alginic acid, aspartic acid, benzoic acid, benzenesulfonic acid, bisulfic acid, butyric acid, citric acid, camphoric acid, camphorsulfonic acid, cyclopentanepropionic acid, digluconic acid, dodecylsulfic acid, ethanesulfonic acid, formic acid, fumaric acid, glucoheptanoic acid, glycerophosphic acid, glycolic acid, hemisulfic acid, heptanoic acid, hexanoic acid, hydrochloride, hydrobromide, hydroiodide, 2-hydroxyethanesulfonic acid, lactic acid, maleic acid, malonic acid, methanesulfonic acid, 2-naphthalenesulfonic acid, nicotinic acid, nitric acid, oxalic acid, palmoic acid, pectinic acid, persulfic acid, 3-phenylpropionic acid, phosphic acid, picric acid, pivalic acid, propionic acid, p-toluenesulfonic acid, salicylic acid, succinic acid, sulfic acid, tartaric acid, thiocyanic acid, and undecanoic acid. Formation of the salt of formula 2 allows for the isolation of the compound without the need for expensive chromatographic purification. Accordingly, formation of the salt is advantageous when the reaction is carried out on a commercial scale.

In one embodiment of the invention, the second reaction mixture is cooled, e.g., to 20±5° C.

During the synthetic transformations described herein, protection of certain functional groups may be desired. The need for such protection will vary depending on the nature of the functional groups and the reaction conditions employed, and can be readily determined by one skilled in the art. For example, according to the methods of the invention, the starting compound of formula 3 can comprise one or more protecting groups, for example on a ring nitrogen or on a functional group present in group B, D, or A. The starting amine of formula R⁴NH₂ can also comprise one or more protecting groups, for example, within the group R⁴. Suitable groups for protecting alcohols, amines, thiols, etc., as well as conditions for attaching and removing such protecting groups, are known to those skilled in the art (See for example, T. W. Greene, Protective Groups in Organic Synthesis, John Wiley & Sons, New York, Third Ed., 1999). Protecting groups can be added to or removed from the intermediate compounds described herein at any synthetically feasible point in the preparation of a compound of formula (I), which points can be readily determined by one working in the field. Accordingly, each step of the methods described herein may further optionally comprise protecting or deprotecting an intermediate compound.

The invention will now be illustrated by the following non-limiting Examples.

### EXAMPLES

### Example 1

### Preparation of a Representative Compound of Formula (I)

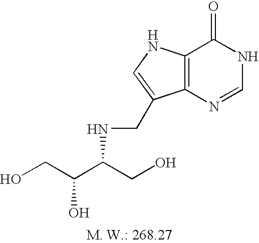

### 7-(((2R,3S)-1,3,4-trihydroxybutan-2-ylamino)methyl)-3H-pyrrolo[3,2-d]pyrimidin-4(5H)-one

A mixture of 4-(benzyloxy)-5H-pyrrolo[3,2-d]pyrimidine (967 g, 4298 mmol) and (5S,6R)-6-amino-2,2-dimethyl-1,3-dioxepan-5-ol acetate (1000 gm, 4525 mmol) in tert-Butanol (11.3 L) was heated at 60±5° C. until the solution became homogeneous. To the homogenous reaction mixture was added formalin (37% aqueous, 403 mL, 4980 mmol) and heated at reflux overnight (16±5 h). A clear solution of L-Tartaric acid (510 g, 3398 mmol) in isopropanol (6780 mL, dissolved by heating) was added to the reaction mixture and cooled to RT (20±5° C.) over a period of four hours. The solid obtained was collected by filtration, washed with isopropanol (3 L) and acetone (6 L). The solid tartrate salt was dried in a vacuum oven at 50° C. for 17±5 h to yield tartarate salt of the desired product (2007 g, yield 85.21%, HPLC purity 93.59% product, 2.42% dimer) as an off white solid; MP 48° C. ¹H NMR (300 MHz, DMSO) δ 12.14 (s, 1H), 8.47 (s, 1H), 7.71 (s, 1H), 7.59-7.50 (m, 2H), 7.46-7.33 (m, 3H), 5.62 (s, 2H), 4.23-4.10 (m, 2H), 4.06 (s, 2H), 3.84-3.72 (m, 1H), 3.59 (dd, J=6.4, 10.9, 2H), 3.45 (d, J=7.2, 2H), 2.65 (s, 1H), 1.25 (s, 3H), 1.23 (s, 3H).

The above tartarate salt (2005 g, 3659 mmol) was taken in ethyl acetate (3600 mL) and to the slurry was added aq. 2.5N NaOH (3600 mL) and stirred until homogeneous. The aqueous layer was separated and extracted with ethyl acetate (2 L and 1 L). The combined organic layers were dried, filtered and concentrated in vacuo to furnish the desired compound as a free base (1516 g, yield 88.6%). ¹H NMR (300 MHz, DMSO) δ 11.61 (s, 1H), 8.14 (d, 1H, J=14.4), 7.31 (d, 2H, J=8.0), 7.14 (m, 3H), 5.36 (s, 2H), 4.54 (d, 1H, J=5.5), 3.67 (q, 2H, J=13.7), 3.41 (dd, 1H, J=2.9, 12.2), 3.20 (m, 4H), 2.97 (s, 1H), 2.16 (d, 1H, J=5.2), 1.84 (s, 1H), 0.97 (s, 6H). IR 3421, 1628, 1535, 1220, 1045. MS (ES+) 399.3 (M⁺¹), (ES−) 397.3 (M⁻¹).

The a solution of above free base (1516 g, 3807 mmol) in ethanol (3.8 L) was added water (1.5 L), conc. HCl (330 mL, 4000 mmol) and heated at gentle reflux for 16±5 h. To the hot reaction mixture was added activated carbon (151.5 g 10% wt) and filtered hot through a pad of Celite. The filtrate was concentrated in vacuo to yield hydrochloride salt of the desired product (1343 g) as oil.

The above crude hydrochloride salt (1343 g) was dissolved in water (1 L) and heated at 60±5° C. To the hot solution was added ethanol (11.55 L, in 1 L intervals slowly, the product self seeds after 4 to 5 L of ethanol addition). The solution was cooled to room temperature overnight and the solid obtained was collected by filtration, washed with ethanol (1.5 L), and dried in vacuo oven at 50° C. for 17±5 h to furnish the desired product (698.6 g, yield 53.27%) as a white solid; MP 230° C. ¹H NMR (300 MHz, DMSO) δ 12.38 (s, 1H), 12.10 (s, 1H), 8.77 (s, 1H), 8.36 (s, 1H), 7.90 (d, J=3.6, 1H), 7.61 (d, J=3.1, 11H), 5.56 (d, J=5.3, 1H), 5.34 (d, J=4.9, 1H), 4.91 (s, 1H), 4.32 (s, 2H), 3.87 (d, J=12.2, 1H), 3.74 (s, 1H), 3.64 (d, J=12.3, 1H), 3.47 (s, 2H), 3.07 (s, 1H). IR (KBr) 3397, 3138, 3089, 3043, 2958, 2842, 1693, 1584, 1445, 1260, 1036, 781, 599 cm⁻¹; MS (ES+1) 269.1 (100%, M+1). HPLC (BCX-5235 method, Zorbax SBC3, 250×4.6 mm, 5 μm, mobile phase: 10 mM Potassium Phosphate, pH 2.5 adjusted with phosphoric acid/Acetonitrile) Rt=7.8904 min, 99.68% and Rt=11.86, 0.32% for dimer; [α]=−20 (c=0.4, H2O)

Analysis: Calcd for C₁₁H₁₆N₄O₄.HCl: C, 43.36; H, 5.62; N, 18.39; Cl, 11.62. Found: C, 43.55; H, 5.65; N, 18.31; Cl, 11.56.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for preparing a compound of formula 2: 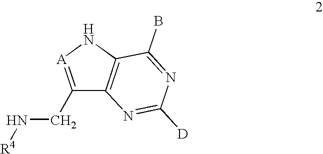
wherein:
R⁴ is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR^{a}R^{b} groups, where each alkylthio, arylthio and aralkylthio group is optionally substituted with one or more alkyl, halogen, amino, hydroxy, or alkoxy groups; or R⁴ is such a group bearing one or more protecting groups;
A is N or CH;
B is OH, or alkoxy;
D is H, OH, NH₂, or SCH₃; and
R^{a} and R^{b} are each independently an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, or nitro;
comprising reacting a corresponding compound of formula 3 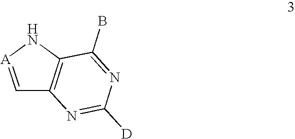
with a corresponding amine of formula R⁴NH₂ and a formaldehyde source in a suitable solvent to provide a reaction mixture comprising the compound of formula 2 in at least about 60% percent yield based on the starting compound of formula 3.

2. A method for preparing a salt of a compound of formula 2: 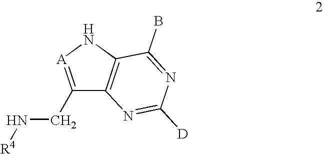
wherein:
R⁴ is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR^{a}R^{b} groups, where each alkylthio, arylthio and aralkylthio group is optionally substituted with one or more alkyl, halogen, amino, hydroxy, or alkoxy groups; or R⁴ is such a group bearing one or more protecting groups;
A is N or CH;
B is OH or alkoxy;
D is H, OH, NH₂, or SCH₃; and
R^{a} and R^{b} are each independently an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, or nitro;
comprising reacting a corresponding compound of formula 3 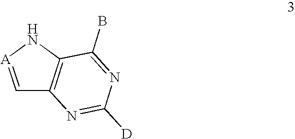
with a corresponding amine of formula R⁴NH₂ and a formaldehyde source in a suitable solvent to provide the corresponding compound of formula 2 in a reaction mixture; and
combining a suitable acid with the reaction mixture to provide the salt of the compound of formula 2 in a second reaction mixture.

3. The method of claim 1, wherein A is CH.

4. The method of claim 1, wherein B is OH.

5. The method of claim 1, wherein D is H.

6. The method of claim 1, wherein R⁴ is alkyl, alkenyl, or alkynyl, which is optionally substituted with one or more hydroxy, alkoxy, thiol, alkylthio, arylthio, aralkylthio, halogen, carboxylic acid, carboxylate alkyl ester, nitro, or NR^{a}R^{b} groups; or R⁴ is such a group bearing one or more protecting groups.

7. The method of claim 1, wherein R⁴ is alkyl, alkenyl, or alkynyl, which is optionally substituted with one or more hydroxy or alkoxy groups; or R⁴ is such a group bearing one or more protecting groups.

8. The method of claim 1, wherein R⁴ is alkyl which is optionally substituted with one or more hydroxy groups; or R⁴ is such a group bearing one or more protecting groups.

9. The method of claim 1, wherein R⁴ is —CH[CH₂OH][CH(OH)CH₂OH]; or R⁴ is such a group bearing one or more protecting groups.

10. The method of claim 1 wherein R⁴ is 2,2-dimethyl-1,3-dioxepan-5-ol-6-yl.

11. The method of claim 1, wherein the formaldehyde source is formalin or paraformaldehyde.

12. The method of claim 1, wherein the formaldehyde source is formalin.

13. The method of claim 1, wherein the solvent comprises an alcohol.

14. The method of claim 1, wherein the solvent comprises a (C₁-C₆)alcohol.

15. The method of claim 1, wherein the solvent comprises tert-butanol or isopropanol.

16. The method of claim 1, wherein the solvent comprises tert-butanol.

17. The method of claim 1, wherein the solvent is tert-butanol.

18. The method of claim 1, wherein the compound of formula 3 is reacted with the amine of formula R⁴NH₂ and the formaldehyde source at a temperature of 70±20° C.

19. The method of claim 1, wherein the compound of formula 3 is reacted with the amine of formula R⁴NH₂ and the formaldehyde source at reflux.

20. The method of claim 2, wherein A is CH.

21. The method of claim 2, wherein B is OH.

22. The method of claim 2, wherein D is H.

23. The method of claim 2, wherein R⁴ is alkyl, alkenyl, or alkynyl, which is optionally substituted with one or more hydroxy, alkoxy, thiol, alkylthio, arylthio, aralkylthio, halogen, carboxylic acid, carboxylate alkyl ester, nitro, or NR^{a}R^{b} groups; or R⁴ is such a group bearing one or more protecting groups.

24. The method of claim 2, wherein R⁴ is alkyl, alkenyl, or alkynyl, which is optionally substituted with one or more hydroxy or alkoxy groups; or R⁴ is such a group bearing one or more protecting groups.

25. The method of claim 2, wherein R⁴ is alkyl which is optionally substituted with one or more hydroxy groups; or R⁴ is such a group bearing one or more protecting groups.

26. The method of claim 2, wherein R⁴ is —CH[CH₂OH][CH(OH)CH₂OH]; or R⁴ is such a group bearing one or more protecting groups.

27. The method of claim 2 wherein R⁴ is 2,2-dimethyl-1,3-dioxepan-5-ol-6-yl.

28. The method of claim 2, wherein the formaldehyde source is formalin or paraformaldehyde.

29. The method of claim 2, wherein the formaldehyde source is formalin.

30. The method of claim 2, wherein the solvent comprises an alcohol.

31. The method of claim 2, wherein the solvent comprises a (C₁-C₆)alcohol.

32. The method of claim 2, wherein the solvent comprises tert-butanol or isopropanol.

33. The method of claim 2, wherein the solvent comprises tert-butanol.

34. The method of claim 2, wherein the solvent is tert-butanol.

35. The method of claim 2, wherein the compound of formula 3 is reacted with the amine of formula R⁴NH₂ and the formaldehyde source at a temperature of 70±20° C.

36. The method of claim 2, wherein the compound of formula 3 is reacted with the amine of formula R⁴NH₂ and the formaldehyde source at reflux.

37. The method of claim 2, wherein the acid is acetic acid, adipic acid, alginic acid, aspartic acid, benzoic acid, benzenesulfonic acid, bisulfic acid, butyric acid, citric acid, camphoric acid, camphorsulfonic acid, cyclopentanepropionic acid, digluconic acid, dodecylsulfic acid, ethanesulfonic acid, formic acid, fumaric acid, glucoheptanoic acid, glycerophosphic acid, glycolic acid, hemisulfic acid, heptanoic acid, hexanoic acid, hydrochloride, hydrobromide, hydroiodide, 2-hydroxyethanesulfonic acid, lactic acid, maleic acid, malonic acid, methanesulfonic acid, 2-naphthalenesulfonic acid, nicotinic acid, nitric acid, oxalic acid, palmoic acid, pectinic acid, persulfic acid, 3-phenylpropionic acid, phosphic acid, picric acid, pivalic acid, propionic acid, p-toluenesulfonic acid, salicylic acid, succinic acid, sulfic acid, tartaric acid, thiocyanic acid, or undecanoic acid.

38. The method of claim 37, wherein the acid is tartaric acid.

39. The method of claim 2, further comprising cooling the second reaction mixture.

40. The method of claim 39, wherein the second reaction mixture is cooled to 20±5° C.

41. The method of claim 2, further comprising isolating the salt of the compound of formula 2.

42. The method of claim 41, further comprising washing the salt of the compound of formula 2 with one or more wash solvents.

43. The method of claim 42, wherein the salt of the compound of formula 2 is washed with isopropanol.

44. The method of claim 42, wherein the salt of the compound of formula 2 is washed with acetone.

45. The method of claim 41, further comprising converting the salt of the compound of formula 2 to the corresponding free-base.

46. The method of claim 45, wherein the salt of the compound of formula 2 is converted to the free-base by treatment with an aqueous base to provide a free-base mixture.

47. The method of claim 46, wherein the base is aqueous sodium hydroxide.

48. The method of claim 46, further comprising extracting the free-base mixture with an organic solvent to provide an organic solvent free-base mixture, and concentrating the organic solvent free-base mixture to provide the isolated free base.

49. The method of claim 48, wherein the organic solvent is ethyl acetate.

50. The method of claim 48, wherein R⁴ is a group bearing one or more protecting groups, further comprising dissolving the isolated free-base in an organic solvent and treating with an acid to remove the one or more protecting groups, and optionally treating with activated carbon, to provide the corresponding acid salt of the compound of formula 2.

51. The method of claim 50, wherein the acid is hydrochloric acid.

52. The method of claim 50, further comprising isolating the corresponding acid salt of the compound of formula 2.

53. The method of claim 52, wherein the corresponding acid salt of the compound of formula 2 is isolated by filtration and concentration.

54. The method of claim 53, further comprising recrystallizing the corresponding acid salt of the compound of formula 2 to provide a final product of formula 2.

55. The method of claim 54, wherein the corresponding acid salt of the compound of formula 2 is recrystallized from water and ethanol.

56. The method of claim 1, wherein at least about 10 grams of the compound of formula 3 is reacted with the amine of formula R⁴NH₂ and the formaldehyde source.

57. The method of claim 1, wherein R⁴ is a group bearing one or more protecting groups, which further comprises removing the one or more protecting groups.

58. The method of claim 2, wherein at least about 10 grams of the compound of formula 3 is reacted with the amine of formula R⁴NH₂ and the formaldehyde source.

59. The method of claim 2, wherein R⁴ is a group bearing one or more protecting groups, which further comprises removing the one or more protecting groups.
